(19) United States
(12) Patent Application Publication
(10) Pub. No.: US 2006/0149131 A1
(43) Pub. Date: Jul. 6, 2006

(54) Surgical tool for endoscope

(75) Inventor: Yakov Bar Or, Haifa (IL)

Correspondence Address: BRUCE E. LILLING;LILLING & LILLING P.C., P.O. BOX 560, GOLDEN BRIDGE, NY, 10526, US

(73) Assignee: Sightline Technologies Ltd., Haifa (IL)

(21) Appl. No.: 11/300,826

(22) Filed: Dec. 14, 2005

Related U.S. Application Data
(60) Provisional application No. 60/641,638, filed on Jan. 5, 2005.

(51) Int. Cl. A61B 1/00

(52) U.S. Cl. 600/154; 600/104

(57) ABSTRACT

Apparatus for sheathing an endoscopic tool is described. The apparatus is suitable for sheathing the tool when it is displaced in a proximal direction from a working channel of an endoscope. The apparatus comprises a sheathing assembly, which is attachable to a proximal port of the endoscope and to the endoscopic tool. Within the sheathing assembly is deployed a folded, flexible sleeve adapted to unfold from the assembly when the tool is retracted in the proximal direction from the working channel. The sheathing assembly is designed to allow displacement of the tool through the sheathing assembly in a distal direction and simultaneous retraction of the tool together with the sheathing assembly in the proximal direction.

### FIELD OF THE INVENTION

The present invention relates generally to endoscopy, and specifically to an endoscopic tool provided with a sleeve which is capable of covering the endoscopic tool during withdrawal of the tool from a body passage.

### BACKGROUND OF THE INVENTION

The use of a disposable sleeve (also referred to as a sheath) to cover an endoscope is well known in the art. Flexible endoscopes, such as colonoscopes, are notoriously difficult to clean and disinfect thoroughly, leading to problems of cross-contamination between patients and between patients and staff. These problems can be partially avoided by covering the endoscope with a single-use sleeve, which is discarded after use.

Endoscopes commonly have working channels, running from a proximal port outside the body to a distal port at the distal end of the endoscope. When the distal end of the endoscope is inserted into the body, the working channel may be used, inter alia, to pass a surgical instrument (further referred-to also as endoscopic tool) through to the distal end of the endoscope in order to perform a surgical procedure, such as a biopsy. Instruments that are used in this manner become contaminated with biological matter from inside the patient's body. As the instrument is withdrawn from the body, it spreads the contamination to the interior of the working channel and to the proximal port of the endoscope and eventually to the operator's hands.

Methods for sheathing an endoscope while providing working channels that protect the endoscope from contamination are described, for example, in Silverstein and Sidall (U.S. Pat. Nos. 4,646,722 and 4,741,326), whose disclosures are incorporated herein by reference. These methods attempt to prevent contamination of the endoscope, either by adding disposable working channels external to the endoscope itself (U.S. Pat. No. 4,646,722) or by adding a disposable liner inside a working channel of the endoscope (U.S. Pat. No. 4,741,326). They do not address the problem, however, of contamination that may be spread to the area around the proximal end of the endoscope as the surgical tool is retracted from the proximal end of the working channel.

### SUMMARY OF THE INVENTION

Embodiments of the present invention provide methods and devices for use in sheathing an endoscopic tool employed in an endoscope having a suitable means to keep the operator's hands clean. The invention is used as the tool is removed from the patient's body. Such sheathing prevents contaminants that may adhere to the endoscopic tool inside the body from contacting the operator's hands, the handle of the endoscope, or other objects outside the patient's body. As a result, the likelihood of cross-contamination between patients and between patients and staff is reduced, and the job of cleaning and disinfecting the endoscope and ancillary equipment between uses is simplified.

In embodiments of the present invention, a sheathing assembly comprising a sleeve dispenser mates with the proximal port of an endoscopic working channel, outside the patient's body. A flexible sleeve is typically fixed by its one end to the dispenser, with the remainder of the sleeve bunched inside or otherwise held in the vicinity of the dispenser. An elongate endoscopic tool is passed through the dispenser and the working channel, until the distal end of the tool protrudes from the distal end of the endoscope. While the shaft of the tool is advanced through the dispenser and the proximal port of the endoscope, the sleeve remains bunched at the dispenser. When the tool is retracted, however, the proximal end of the sleeve engages the shaft of the tool, so that as the tool is withdrawn, the sleeve unfurls from the dispenser to cover the shaft of the tool, up to and including its distal end. All contaminants on the tool thus remain within the sleeve, while the outside of the sleeve remains clean and can be handled freely without spreading contamination.

Embodiments of the present invention are particularly (although not exclusively) suited for use with the endoscopes, which are provided with sheathing systems for covering the outer surface of an endoscope, such as the systems described in Eizenfeld (WO 2004/016299-PCT/IL03/000661), which is incorporated herein by reference, or endoscopes provided with other sheathing systems known in the art. Such sheathing systems typically include disposable working channels or a disposable internal sleeve that fits inside the working channel of the endoscope and protects it from contamination. Endoscope sheaths of this sort cover the endoscope while the endoscope is inside the patient's body, so that the endoscope remains clean.

The sheath is removed thereafter, and the endoscope proceeds to intermediate disinfection. There is also known in the art an apparatus for sheathing an endoscopic tool as described in Aizenfeld (U.S. Pat. No. 6,908,428), which disclosure is incorporated herein by reference.

The sheathing techniques employed in the apparatus described in this patent are used to cover the endoscopic tool outside the body, after it has been withdrawn from the working channel. Optionally, if required during the surgical procedure, the tool may subsequently be advanced again distally out of the sleeve and into the working channel, and afterwards retracted back into the sleeve. When the surgical procedure is finished, the tool and the sleeve are disposed of together.

The apparatus for sheathing the endoscopic tool, in accordance with an embodiment disclosed in the above-referred to patent includes: a sheathing assembly, including a sleeve dispenser and a port adapter for mating with a proximal port of an endoscopic working channel so as to permit the endoscopic tool to be advanced through the sheathing assembly into the working channel; and a flexible sleeve, including a distal end, which is fixed to the sleeve dispenser, and a proximal end, which is adapted to engage the endoscopic tool as the endoscopic tool is retracted from the working channel, causing the sleeve to extend out of the dispenser in a proximal direction so as to sheath the endoscopic tool.

In an additional embodiment, the tool is adapted to extend through the working channel in order to capture biological matter within a body of a patient, and, after retraction of the endoscopic tool from the working channel, the sleeve dispenser is adapted to communicate with a container so as to permit the endoscopic tool to release the biological matter into the container while the endoscopic tool remains sheathed with the sleeve.

The sheathing assembly disclosed in this patent is in fact a separate, stand-alone item, which is dedicated for sheathing the endoscopic tool when it is being evacuated from the endoscope. The assembly should be attached to the endoscopic tool before use and then it can be secured on a port of the endoscope before insertion the tool in the working channel. The assembly is designed to stay on the port and to enable the sleeve to retain its bunched state while the tool protracts through the assembly and to engage the tool and to unfurl when the tool retracts from the assembly.

In practice this solution is complicated, since it necessitates securing a separate item on the endoscope before protracting the endoscopic tool into working channel.

Furthermore, the sheathing assembly is designed in such a manner that, when the sleeve unfurls, it escapes the sheathing assembly alone, while the dispenser remains to be connected to the endoscope's proximal port. This specific design renders the manual grasping of the tool by the doctor's hand inconvenient. Furthermore, feeding out of the sleeve alone may be unstable and this may complicate the retraction of the tool from the working channel.

The present invention seeks to provide a solution, which is free from the above mentioned disadvantages of the known in the art sheathing system, yet, however retains its advantages.

One object of the present invention is providing a combination of a sheathing assembly with an endoscopic tool, said combination being an integral, sole, dedicated item, which is ready for use and whose operation is simple and convenient.

Another object of the present invention is providing a new sheathing assembly, which enables convenient grasping of the endoscopic tool by the operator's hand during evacuation of the tool from the endoscope.

Still a further object of the present invention is providing a sheathing assembly, which ensures stable and reliable feeding out of the sleeve during retraction of the tool.

The present invention concerns also a new sheathing assembly and a new method of sheathing the endoscopic tool by a disposable sleeve.

For a better understanding of the present invention as well of its benefits and advantages, reference will now be made to the following description of its embodiments taken in combination with the accompanying drawings.

### BRIEF DESCRIPTION OF THE DRAWINGS

FIG. 1 is a schematic illustration of a combination of the sheathing assembly with the endoscopic tool in accordance with an embodiment of the present invention;

FIG. 2 is a cross-sectional, enlarged view of the combination shown in FIG. 1;

FIG. 2a is a side view of the sheathing assembly shown in FIG. 2;

FIG. 2b is a cross-sectional, enlarged view of the sheathing assembly shown in FIG. 2;

FIG. 3 is a schematic illustration showing an initial stage of connecting the sheathing assembly to a proximal port of an endoscope;

FIG. 4 is a schematic illustration showing the sheathing assembly fitted to the proximal port of the endoscope;

FIG. 5 is a schematic illustration showing an initial stage of protracting the endoscopic tool through the proximal port of the endoscope;

FIG. 6 is a schematic illustration showing disconnection of a rear portion of the sheathing assembly from a frontal portion thereof;

FIG. 7 is a schematic illustration showing an initial stage of removal of the endoscopic tool from the endoscope;

FIG. 8 is a schematic, enlarged illustration showing the rear portion of the sheathing assembly being disconnected from the frontal portion thereof;

FIG. 9 is a schematic illustration showing a final stage of removal of the endoscopic tool from the endoscope; and

FIG. 10 is schematic illustration showing the frontal portion of the sheathing assembly disconnected from the proximal port of the endoscope.

### DETAILED DESCRIPTION OF THE INVENTION

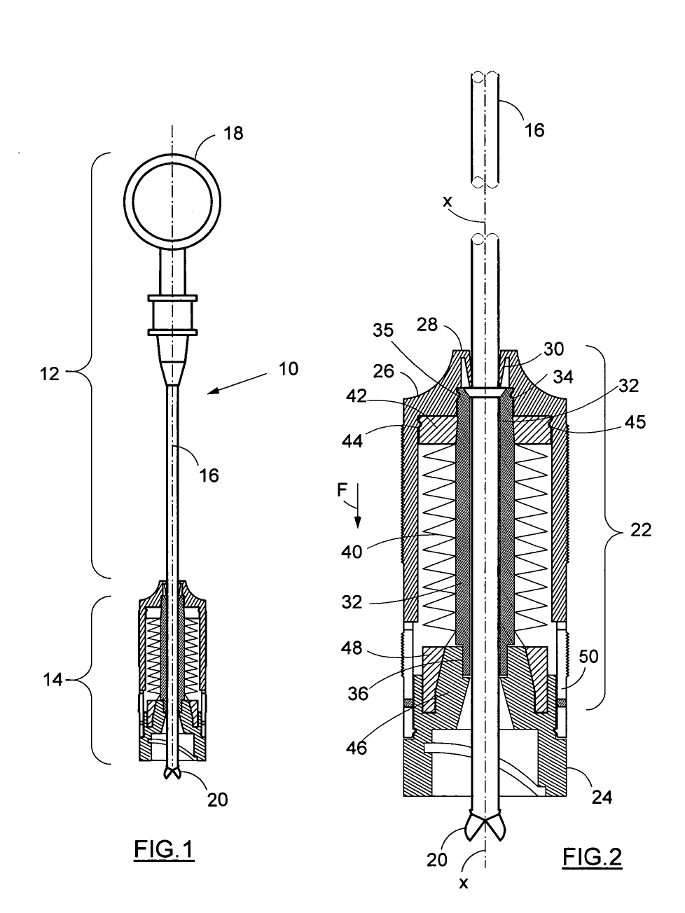

FIG. 1 is a schematic, pictorial illustration of an embodiment of the present invention, which is designated by reference numeral 10. This embodiment refers to a combination of a surgical tool 12 with a sheathing assembly 14. The surgical tool consists of an elongated shaft 16, which can be made of metallic or plastic material. A rear end of the shaft is provided with a thumb ring 18 for operating the tool, while a distal end is provided with a working element 20 for performing a surgical procedure. The working element shown in FIG. 1 is a biopsy forceps, which are operable to take a tissue sample within the patient's body. It should be appreciated, however, that this is only an example and that the working element can be of any other kind as might be required for performing the surgical procedure, e.g. a polypectomy snair, etc.

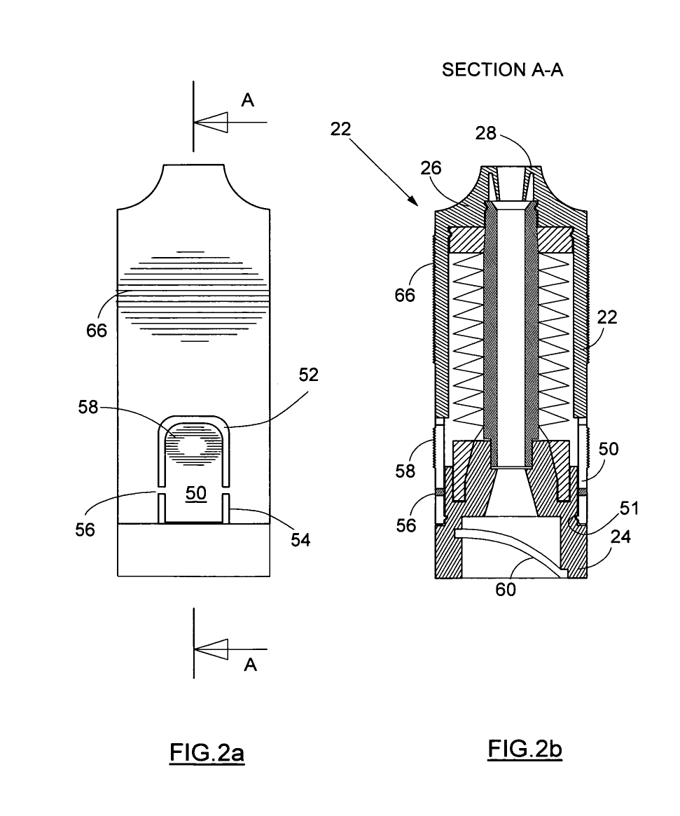

Construction of the sheathing assembly will be explained now in more details with reference to FIGS. 2, 2a, and 2b. The sheathing assembly comprises several separable parts, which function as described hereinbelow. The sheathing assembly comprises a rear, sleeve dispenser portion 22 and releasably connected thereto a frontal portion 24. The rear portion is intended for accommodating therein a covering sleeve, which is intended for protecting the surgical tool, while the frontal portion is intended for fitting to a proximal port of an endoscope. The sheathing assembly is manufactured from suitable rigid plastic material, e.g. PVC, polycarbonate, polystyrene, nylon 66 etc.

The sleeve dispenser portion has a proximal extremity 26 through which the shaft 16 of the surgical tool passes. The proximal extremity is formed with a plug portion 28, whose tapered wall 30 defines an aperture than fits snugly around the shaft. The plug is configured and dimensioned in such a manner that it allows entering the shaft through the aperture and displacement of the shaft with little resistance only in a forward (distal) direction, e.g. into the patient's body. At the same time the plug prevents the shaft from being displaced in a backward (proximal) direction. In FIG. 2 the forward direction of the shaft is designated by an arrow F. When the tool is retracted in the backward direction the plug catches on the shaft by the tapered wall, thus causing the dispenser portion to be pulled out simultaneously with the surgical tool, as will be shown in the figures that follow. Alternatively, other types of one-way plugs may be used, as will be apparent to those skilled in the art.

By virtue of the plug the sheathing assembly could be coupled to the surgical tool before performing the surgical procedure so that their combination would constitute a stand alone, ready for use, sole item.

Extending coaxially with a longitudinal axis X-X of the sheathing assembly a guiding tube 32 is provided. This tube guides the shaft of the surgical tool, when it passes through the aperture of the conical plug and when it displaces within the sheathing assembly. The tube extends within the dispenser portion between the plug and frontal portion 24. On a proximal end of the guiding tube a circular groove 34 is formed, which is in snapping engagement with a corresponding circular protrusion 35 made in the proximal extremity of the rear portion. By virtue of this provision the tube is secured within the sleeve dispenser portion 26. The distal end of the guiding tube is provided with a neck portion 36.

The sheathing assembly holds within it a flexible sleeve 40, which is initially bunched inside the dispenser portion between a retaining ring 42 and the frontal portion 24. The protecting sleeve can be made of a flexible, biocompatible plastic, such as polyamide, and preferably having a thickness of about 20 microns. The retaining ring is located within the dispenser portion as shown in FIG. 2 and it is held therein by virtue of a circular groove 44, which is in snapping engagement with a corresponding circular protrusion 45 made at the proximal extremity of the dispenser portion. The retaining ring fits snugly around the guiding tube so that the sleeve's proximal end is anchored therebetween.

Frontal portion 24 of the sheathing assembly is provided with a skirt 46 and a cap member 48, which is disposed around the skirt to provide a circular gap therebetween. Cup member 48 fits snugly around the skirt extremity so that the sleeve's distal end is anchored therebetween. The frontal portion of the sheathing assembly is detachably coupled to the rear portion by a few springy lugs 50, which are provided in the forward most end of the rear portion. One such lug is shown in FIG. 2a and it is seen that the lug is confined between two separate cut-outs 52, 54 made in the sleeve dispenser portion. A continuous narrow region 56 divides between the cut-outs and allows the lug to pivot elastically when a lateral pressure is applied to a dedicated roughed region 58 on the lug. It is also seen in FIGS. 2,2b that the distal end of each lug is provided with a protrusion 51 which catches on a circular groove made in the circular wall of the frontal portion 24. By virtue of this provision the rear and the frontal portion of the sheathing assembly remain connected due to the snapping engagement. This engagement terminates when lateral pressure is applied to the lugs.

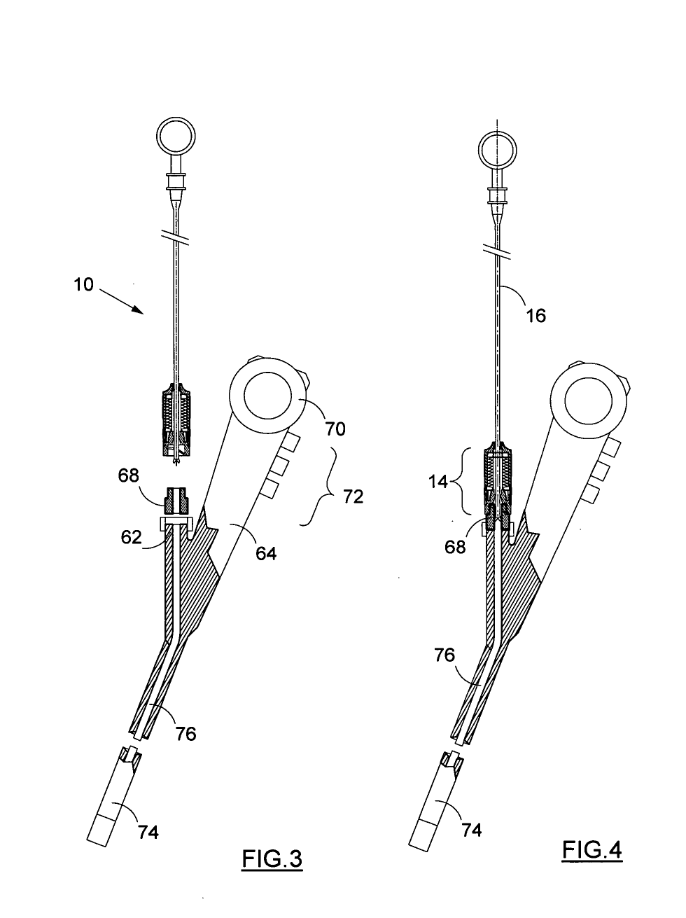

Extending along the inwardly facing surface of the frontal portion a helical groove 60 is provided. This groove is part of a bayonet coupling, which, as will be apparent further with reference to FIGS. 3-10, allows fitting the sheathing assembly onto a port 62 made in a handle 64 of an endoscope, e.g. a colonoscope.

It would be advantageous if the outwardly facing surface of the rear portion 22 is provided with a roughed region 66 as shown in FIG. 2a. This roughed provides a reliable grip and ensures convenient manipulation of the sheathing assembly during its operation.

In FIG. 2 is shown the combination of the sheathing assembly and the surgical tool when it is ready for use. It is seen that the sheathing assembly is located proximate the forwardmost end of the shaft, while working element 20 protrudes from the sheathing assembly being ready for insertion into endoscope.

Referring now to FIG. 3, it shows the situation when the combination of the sheathing assembly and the surgical tool is brought near port 62 and is ready for fitting onto the port either directly or through a dedicated connector 68.

Typically the handle is provided also with a conventional wheel 70 for angulation control and with buttons 72 for control of video signal and for supplying suction, gas pressure and/or irrigation. It is not shown specifically, but should be appreciated, that the handle comprises a suitable fluid port, for applying suction, gas pressure and/or irrigation through the working channel. This is requested when the tool is not inserted in the working channel.

The endoscope has an insertion tube 74, which may be covered by a disposable sheath (not shown). Along the insertion tube extends a working channel 76, through which the surgical tool can be inserted. The working channel may likewise be internally sheathed by a disposable working channel or so-called multilumen tubing, which protects the endoscope's interior from contamination. Such sheathing is described for example in Eizenfeld (WO 2004/016299-PCT/IL03/000661), which is herein incorporated by reference. It should be appreciated that the sheathing is omitted from FIG. 1, for the sake of simplicity.

Additionally or alternatively, additional ports (not shown) can be provided in the handle, for mating with additional channels within or alongside the endoscope.

Since at least the working element of the surgical tool comes into contact with tissue and other biological matter inside the patient's body or in the working channel, the tool necessarily becomes contaminated during use. In order to prevent the spread of contamination from the surgical tool 12 to the handle 64, to the operator's hands and to other areas outside the patient's body, the sheathing assembly is fitted onto port 62 as depicted in FIGS. 4-6. Sheathing assembly 14 mates with port 62 via connector 68 so that a passage through the guiding tube and connector is aligned with working channel 76. Typically the sheathing assembly could be secured on the port by a bayonet coupling provided with a pin interacting with helical groove in the frontal portion of the sheathing assembly when the sheathing assembly is twisted.

FIG. 4 shows a sheathing assembly being secured on the port and surgical tool 12 is ready for insertion through working channel 76. As seen in FIG. 5 the tool has been advanced along the working channel in the distal direction, shown by an arrow 78, until its working end 20 protrudes from the distal end of the endoscope so as to be used in the usual manner. It should be appreciated that sheathing assembly 14 may similarly be adapted to mate with a disposable working channel.

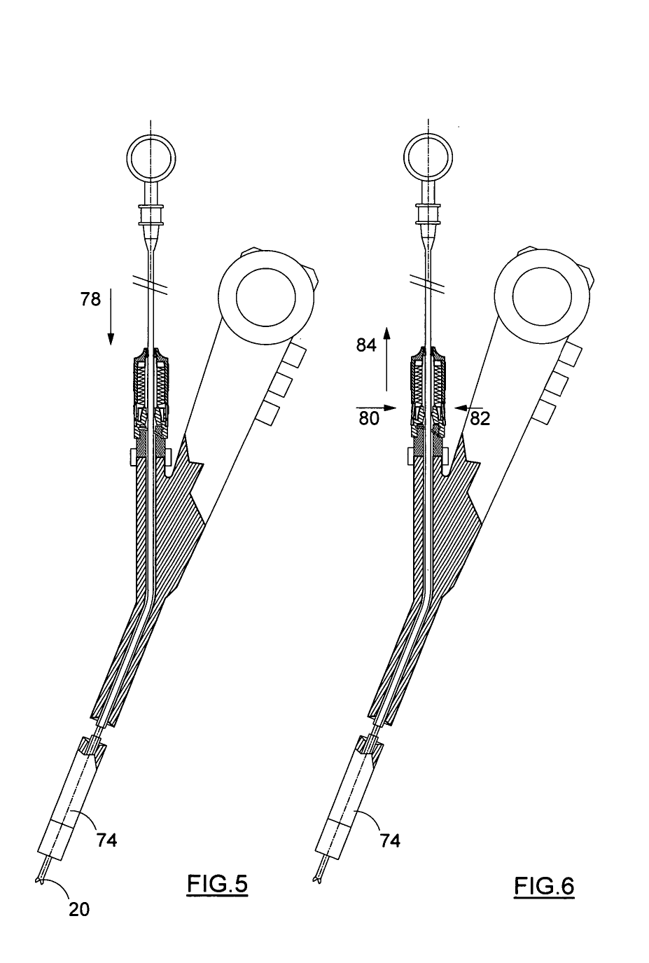

FIG. 6 is a schematic, pictorial illustration showing in accordance with an embodiment of the present invention the beginning of retraction of the surgical tool 12 through sheathing assembly 14. To make this possible, the operator should first manually apply lateral pressure to lugs 50 as shown by arrows 80, 82 so as to detach the lugs from the circular groove on the frontal portion. Then, by grasping the rear portion, the operator should pull the assembly in the proximal direction shown by an arrow 84, so as to detach the rear portion of the assembly from the frontal portion. Seeing that rear portion 22 and the tool displace simultaneously, the detachment can be carried out easily and conveniently.

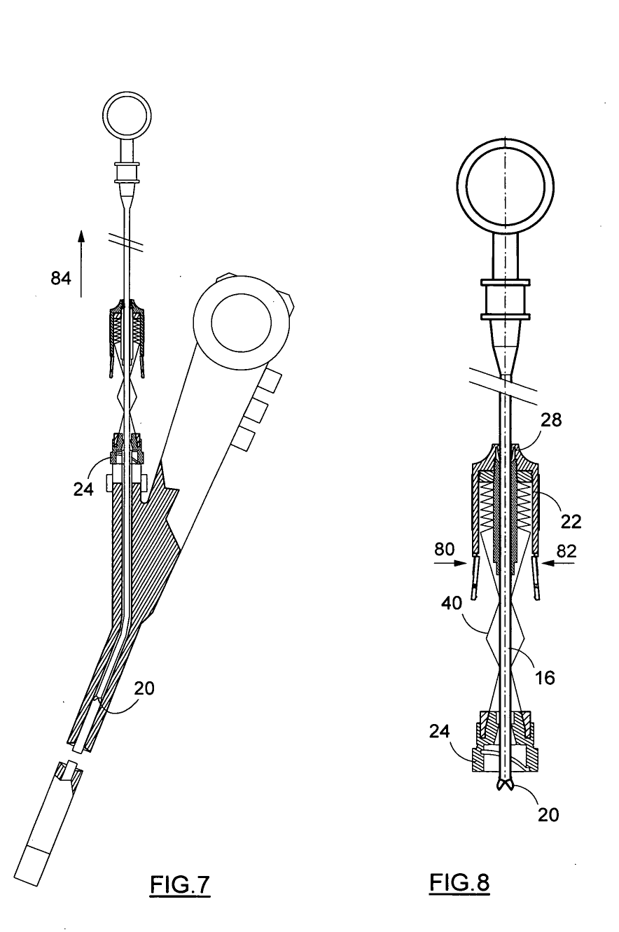

The initial stage of detachment is shown in FIG. 7, while in FIG. 8 is depicted the rear portion after it has been brought remote enough from the frontal portion and working element 20 of the tool approaching frontal portion 24.

It can be readily appreciated that since plug portion 28 grasps shaft 16 as the tool is retracted in the proximal direction, the tool is being retracted simultaneously with the rear portion and with the anchored thereto proximal end of sleeve 40.

Since the distal end of the sleeve remains anchored to frontal portion 24, the sleeve unfurls gradually and covers shaft 16 of the tool. Typically, while the tool is being retracted, the plug portion remains coupled to shaft 16 at the most proximal point on the shaft that the plug portion reached as the tool has been advanced into assembly 14. Therefore, as tool 12 is retracted, the entire length of shaft 16 that previously entered working channel 76 is sheathed by the sleeve up to and including working element 20. This situation is shown in FIG. 9. The sleeve unfurls and feeds out evenly and reliably since its yet unfurled portion remains seated on the guiding tube.

If desired, after the tool has been retracted (in whole or in part), it may repeatedly be advanced into the working channel without removing the tool from sleeve 40, and then retracted again thereafter. In this situation, the sleeve continues to cover any portion of shaft 16 that was previously inserted into the working channel (and is therefore contaminated), but now extends out of the dispenser in the proximal direction.

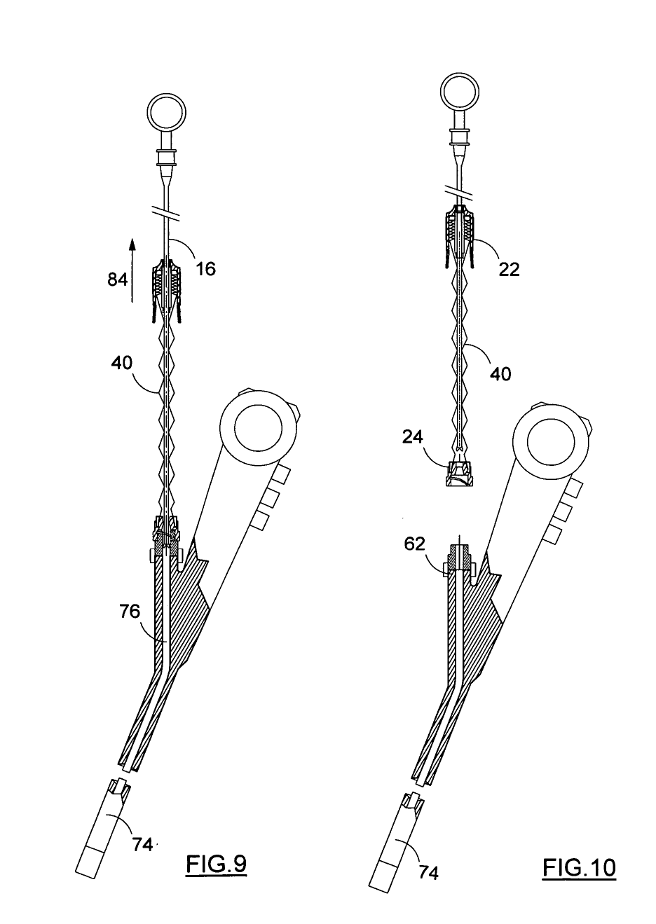

Once tool 12 has been completely retracted from the endoscope and, simultaneously, sheathed by sleeve 40, frontal portion of the sheathing assembly may be detached from port 62 as shown in FIG. 10 and then disposed of along with the tool.

It should be kept in mind that after the first use the sheathing assembly can be used again, however only with the same patient. After this the assembly and the tool should be discarded.

It is not shown in details, however it should be appreciated that the present combination of the sheathing assembly with the surgical tool would be suitable for transfer of a tissue sample from working element to a sample container as it is described in Aizenfeld (U.S. Pat. No. 6,908,428). In accordance with this embodiment tool 12 is to be retracted completely from endoscope and rear portion of sheathing assembly 14 has to be detached from port 62 as seen in FIG. 10. Then dispenser portion should be coupled to the neck of a collecting container (not shown) in the same way as it has been coupled to the frontal portion. At this point, assuming working element 20 to comprise biopsy forceps holding a biopsy sample captured inside the patient's body, for example, the operator of tool 12 may advance element 20 into the neck of container, and may then open the forceps, releasing the biopsy sample into the container. Thus, working element 20 and the sample that it captures are never exposed to the environment outside sleeve 40 and container.

The present invention is particularly suited for use with flexible endoscopes that are inserted into the gastrointestinal tract, such as colonoscopes and gastroscopes, which have relatively large working channels. Alternatively, the principles of the present invention may be applied to sheath tools that are inserted through lumens in medical probes of other types, such as endoscopes (both rigid and flexible) and catheters used in other body passages and in other therapeutic and diagnostic procedures.

It will thus be appreciated that the embodiments described above are cited by way of example, and that the present invention is not limited to what has been particularly shown and described hereinabove. Rather, the scope of the present invention includes both combinations and sub combinations of the various features described hereinabove, as well as variations and modifications thereof which would occur to persons skilled in the art upon reading the foregoing description and which are not disclosed in the prior art.

So for example, the present invention would be suitable for use with endoscopic tools employed either in rigid or resilient endoscope.

Instead of retaining the bunched sleeve within the rear portion as shown in FIG. 2 it could be deployed within a frontal portion that is adapted for securing on the port. In this situation pulling the rear portion in the proximal direction will cause the sleeve to feed out of the frontal portion and to sheath the tool.

Furthermore, instead of using springy lugs for keeping the rear portion detachably coupled to the frontal portion, one could use any other known in the art arrangement, e.g. snap connection.

Furthermore, the guiding tube can extend beyond the frontal portion so as to protrude into the proximal port. By virtue of this provision it would be possible to prevent contamination of the proximal port.

I claim:

1. A combination of a sheathing assembly and an endoscopic tool for sheathing the endoscopic tool, said sheathing assembly being attachable to a proximal port of the endoscope and to the endoscopic tool, said sheathing assembly accommodating therein a folded, flexible sleeve unfolding from the assembly to sheath the endoscopic tool as it is being retracted, wherein said combination allows displacement of the endoscopic tool through the sheathing assembly in a distal direction and simultaneous retraction of the endoscopic tool together with the sheathing assembly in a proximal direction.

2. The combination as defined in claim 1, wherein said combination further comprises a means for attachment the sheathing assembly to the endoscopic tool, said means for attachment comprising a one-way plug fitting snugly around a shaft of the endoscopic tool to allow displacement of the shaft with little resistance in the distal direction, while preventing the shaft from displacing with respect to the plug in the proximal direction.

3. The combination as defined in claim 2, wherein said combination further comprises a means for attachment the sheathing assembly to a proximal port of the endoscope, said means for attachment comprising a bayonet.

4. The combination as defined in claim 2, wherein said sheathing assembly comprises frontal portion and a rear, sleeve dispenser portion, which accommodates therein the flexible sleeve, said sleeve dispenser portion being releasable connectable to the frontal portion.

5. The combination as defined in claim 4, wherein said sheathing assembly comprises a guiding tube, which extends between the plug and the frontal portion, said guiding tube enabling deployment thereon and unfolding therefrom of the folded sleeve.

6. The combination as defined in claim 5, wherein said sheathing assembly is provided with springy lugs, which allow disconnection of the rear, sleeve dispenser portion from the frontal portion.

7. The combination as defined in claim 4, wherein said one-way plug is an integral part of the rear, sleeve dispenser portion.

8. An apparatus for sheathing an endoscopic tool when it is displaced in a proximal direction, said apparatus comprising:
a sheathing assembly attachable to the endoscopic tool, and said sheathing assembly having means for allowing displacement of the endoscopic tool through the sheathing assembly in a distal direction and simultaneous retraction of the endoscopic tool together with the sheathing assembly when the endoscopic tool is being displaced in the proximal direction; and
a folded, flexible sleeve deployed in the sheathing assembly and unfolding from the assembly to sheath the endoscopic tool as it is being retracted in the proximal direction from the working channel.

9. The apparatus for sheathing as defined in claim 8, wherein said sheathing assembly comprises a frontal portion and a rear, sleeve dispenser portion, which accommodates therein the flexible sleeve; the frontal portion attachable to the proximal port of the endoscope, said sleeve dispenser portion being releasably connectable to the frontal portion.

10. The apparatus for sheathing as defined in claim 9, wherein said rear, sleeve dispenser portion is provided with a means for attachment of the sheathing assembly to the endoscopic tool, said means for allowing displacement comprising a one-way plug fitting snugly around a shaft of the endoscopic tool to allow displacement of the shaft with little resistance in the distal direction, while preventing the shaft from displacing with respect to the plug when it is being retracted in the proximal direction.

11. The apparatus for sheathing as defined in claim 10, wherein said sheathing assembly comprises a guiding tube, which extends between the plug and the frontal portion, said guiding tube enabling deployment thereon and unfolding therefrom of the folded sleeve.

12. The apparatus for sheathing as defined in claim 11, wherein said sleeve has a proximal end anchored in the rear, sleeve dispenser portion and a distal end anchored in the frontal portion.

13. The apparatus for sheathing as defined in claim 12, wherein said one-way plug is an integral part of the rear, sleeve dispenser portion.

14. The apparatus for sheathing as defined in claim 12, wherein said sheathing assembly is provided with springy lugs allowing disconnection of the rear, sleeve dispenser portion from the frontal portion.

15. The apparatus for sheathing as defined in claim 12, wherein said frontal portion is provided with a means for attachment of the sheathing assembly to a proximal port of the endoscope, said means for attachment comprising a bayonet.

16. A method for sheathing an endoscopic tool when it is displaced in a proximal direction, said method comprises:
attaching a frontal portion of a sheathing assembly to a proximal port of the endoscope, said sheathing assembly accommodating therein a folded, flexible sleeve unfolding from the assembly to sheath the endoscopic tool as it is being retracted from the working channel,
securing a rear, sleeve dispenser portion of the sheathing assembly on a shaft of the endoscopic tool,
disconnecting of the rear portion from the frontal portion; and
simultaneous retraction of the endoscopic tool together with the rear portion of the sheathing assembly in the proximal direction.

17. The method for sheathing as defined in claim 16, wherein said attaching the frontal portion of the sheathing assembly to the proximal port is carried out by a bayonet.

18. The method for sheathing as defined in claim 16, wherein said securing the rear, sleeve dispenser portion of the sheathing assembly on the shaft of the endoscopic tool comprises using a one-way plug which allows displacing of the shaft with little resistance in the distal direction, while preventing the shaft from displacing with respect to the plug in the proximal direction.

19. The method for sheathing as defined in claim 16, wherein said disconnecting of the rear portion from the frontal portion is carried out by pressing on springy lugs.

20. The method for sheathing as defined in claim 16, comprising deployment of the folded, flexible sleeve on a guiding tube, which extends between the plug and the frontal portion, said guiding tube enabling unfolding therefrom of the folded sleeve.
